(19) United States
(12) Patent Application Publication
(10) Pub. No.: US 2026/0211098 A1
(43) Pub. Date: Jul. 23, 2026

(54) ESTIMATING DEVICE, ESTIMATING METHOD, AND RECORDING MEDIUM

(71) Applicant: Panasonic Intellectual Property Management Co., Ltd., Osaka (JP)

(72) Inventors: Shoichi IIZUKA, Osaka (JP); Takeshi NAKAYAMA, Hyogo (JP); Nobuyuki SHIRAKI, Hokkaido (JP); Naoki HONMA, Iwate (JP)

(21) Appl. No.: 19/143,106

(22) PCT Filed: Dec. 19, 2023
(86) PCT No.: PCT/JP2023/045511

§ 371 Date: Jun. 25, 2025

(30) Foreign Application Priority Data
Dec. 27, 2022 (JP) 2022-209282

(51) Int. Cl. G01S 13/10; G01S 7/285

(52) U.S. Cl. CPC G01S 13/10; G01S 7/285

(57) ABSTRACT

An estimating device includes: a transmission signal generator; a transmission antenna; a transmitter; a reception antenna; a receiver; a complex transfer function calculator; a third complex transfer function calculator that calculates a third complex transfer function by correcting frequency phase errors and antenna phase errors, from a second complex transfer function calculated by dividing all elements of a first complex transfer function by a direct wave component; a living body correlation matrix calculator; and an estimator that estimates a third distance that is a sum of a first distance between the transmission antenna and the living body and a second distance between the reception antenna and the living body, and a first angle that is a direction of the living body as seen from the transmission antenna or the reception antenna.

### TECHNICAL FIELD

The present disclosure relates to an estimating device, an estimating method, and so on, for estimating the distance or position of a living body by using radio signals.

### BACKGROUND ART

A method that uses radio signals is being considered as a method for knowing the position of a person (see for example, Patent Literature (PTL) 1 to 4). PTL 1, 2, and 3 disclose techniques of estimating the position and state of a person that is a detection target by analyzing a component including a Doppler shift using difference calculation. PTL 4 and 5 disclose Doppler sensors that use orthogonal frequency division multiplexing (OFDM) signals.

### CITATION LIST

### Patent Literature

[PTL 1] Japanese Unexamined Patent Application Publication No. 2015-117972
[PTL 2] Japanese Unexamined Patent Application Publication No. 2017-129558
[PTL 3] Japanese Unexamined Patent Application Publication No. 2018-008021
[PTL 4] Japanese Unexamined Patent Application Publication No. 2012-088279
[PTL 5] Japanese Unexamined Patent Application Publication No. 2012-137340

### Non Patent Literature

[NPL 1] H. Yamada, M. Ohmiya, Y. Ogawa and K. Itoh, “Superresolution techniques for time-domain measurements with a network analyzer,” in IEEE Transactions on Antennas and Propagation, vol. 39, no. 2, pp. 177-183, February 1991

### SUMMARY OF INVENTION

### Technical Problem

With the conventional methods, it is difficult to more accurately estimate the distance from the estimating device to a living body and the direction from the estimating device to the living body.

The present disclosure is conceived in view of the above-described circumstances, and provides an estimating device, and so on, capable of more accurately estimating the distance from the estimating device to a living body and the direction from the estimating device to the living body.

### Solution to Problem

In order to achieve the above object, an estimating device according to one aspect of the present disclosure is a device that estimates a distance to a living body and an angle, and includes: a transmission signal generator that generates a multicarrier signal obtained by modulating S subcarrier signals, where S is a natural number greater than or equal to 2; a transmission antenna including M transmission antenna elements, where M is a natural number greater than or equal to 1; a transmitter that causes the transmission antenna to transmit the multicarrier signal, by processing and outputting the multicarrier signal to the transmission antenna; a reception antenna including N reception antenna elements, where N is a natural number greater than or equal to 1, where at least one of M or N is greater than or equal to 2; a receiver that measures, for a first period equivalent to a cycle derived from an activity of the living body, a plurality of reception signals which are received by each of the N reception antenna elements and include a reflected signal which is the multicarrier signal transmitted from each of the M transmission antenna elements that has been reflected or dispersed by the living body; a first complex transfer function calculator that calculates, for each of S subcarriers to which the S subcarrier signals correspond, a plurality of first complex transfer functions indicating a propagation characteristic between a transmission antenna element and a reception antenna element in each of M×N combinations which are combinations of each of the M transmission antenna elements and each of the N reception antenna elements, using the plurality of reception signals measured by the receiver in the first period, and records the plurality of first complex transfer functions sequentially in time series which is an order in which the plurality of reception signals are measured; a second complex transfer function calculator that calculates a second complex transfer function by dividing all elements based on the plurality of first complex transfer functions by a direct wave component extracted using one or more elements based on the plurality of first complex transfer functions, the direct wave component having arrived not via the living body from the plurality of reception signals; a third complex transfer function calculator that calculates, from the second complex transfer function, a third complex transfer function by correcting, with respect to a reference phase calculated from a positional relationship between the M transmission antenna elements and the N reception antenna elements, an frequency phase error in the S subcarriers of the second complex transfer function, and an antenna phase error in the M transmission antenna elements; a living body correlation matrix calculator that calculates a living body correlation matrix having M×N rows and columns for each of the S subcarriers, by extracting a component related to the living body from the third complex transfer function; and an estimator that estimates, using the living body correlation matrix calculated for each of the S subcarriers, a third distance that is a sum of a first distance between the transmission antenna and the living body and a second distance between the reception antenna and the living body, and a first angle that is a direction of the living body as seen from the transmission antenna or the reception antenna.

An estimating method according to another aspect of the present disclosure is a method of estimating a distance to a living body and an angle, and includes: generating a multicarrier signal obtained by modulating S subcarrier signals, where S is a natural number greater than or equal to 2; causing a transmission antenna to transmit the multicarrier signal, by processing and outputting the multicarrier signal to the transmission antenna, the transmission antenna including M transmission antenna elements, where M is a natural number greater than or equal to 1; measuring, for a first period equivalent to a cycle derived from an activity of the living body, a plurality of reception signals which are received by each of N reception antenna elements included in a reception antenna and which include a reflected signal which is the multicarrier signal transmitted from each of the M transmission antenna elements that has been reflected or dispersed by the living body, where N is a natural number greater than or equal to 1, where at least one of M or N is greater than or equal to 2; calculating, for each of S subcarriers to which the S subcarrier signals correspond, a plurality of first complex transfer functions indicating a propagation characteristic between a transmission antenna element and a reception antenna element in each of M×N combinations which are combinations of each of the M transmission antenna elements and each of the N reception antenna elements, using the plurality of reception signals measured in the first period, and recording the plurality of first complex transfer functions sequentially in time series which is an order in which the plurality of reception signals are measured; calculating a second complex transfer function by dividing all elements based on the plurality of first complex transfer functions by a direct wave component extracted using one or more elements based on the plurality of first complex transfer functions, the direct wave component having arrived not via the living body from the plurality of reception signals; calculating, from the second complex transfer function, a third complex transfer function by correcting, with respect to a reference phase calculated from a positional relationship between the M transmission antenna elements and the N reception antenna elements, a frequency phase error in the S subcarriers of the second complex transfer function, and an antenna phase error in the M transmission antenna elements; calculating a living body correlation matrix having M×N rows and columns for each of the S subcarriers, by extracting a component related to the living body from the third complex transfer function; and estimating, using the living body correlation matrix calculated for each of the S subcarriers, a third distance that is a sum of a first distance between the transmission antenna and the living body and a second distance between the reception antenna and the living body, and a first angle that is a direction of the living body as seen from the transmission antenna or the reception antenna.

It should be noted that these general and specific aspects may be implemented using a system, an integrated circuit, a computer program, or a computer-readable recording medium such as a CD-ROM, or any combination of an apparatus, a system, a method, an integrated circuit, a computer program, or a recording medium.

### Advantageous Effects of Invention

According to the present disclosure, it is possible to more accurately estimate the distance from the estimating device to a living body and the direction from the estimating device to the living body.

### BRIEF DESCRIPTION OF DRAWINGS

FIG. 1 is a block diagram illustrating an example of a configuration of an estimating device according to Embodiment 1.

FIG. 2 is a diagram illustrating an example of a detection target of the estimating device illustrated in FIG. 1.

FIG. 3 is a schematic diagram illustrating that the phase of a reception signal changes due to frequency and distance.

FIG. 4 is a schematic diagram illustrating the relationship between a phase error and a channel according to Embodiment 1.

FIG. 5 is a schematic diagram illustrating the relationship between frequency and phase difference slope.

FIG. 6 is a schematic diagram illustrating phases of a time domain living body component transfer function matrix according to Embodiment 1.

FIG. 7 is a diagram illustrating an example of a positional relationship of a detection target of the estimating device illustrated in FIG. 1.

FIG. 8 is a diagram illustrating an example of positional relationships of detection targets of the estimating device illustrated in FIG. 1, when a plurality of living bodies are present.

FIG. 9 is a flowchart illustrating the estimation process by the estimating device according to Embodiment 1.

### DESCRIPTION OF EMBODIMENTS

### Underlying Knowledge Forming Basis of the Present Disclosure

A method that uses radio signals is being considered as a method for knowing the position of a person.

For example, PTL 1 and 2 disclose transmitting a radio signal over a predetermined area, receiving, using antennas, the radio signal reflected by a detection target, and estimating a complex transfer function between transmission and reception antennas. A complex transfer function is a function of a complex number representing a relationship between input and output, and, here, represents propagation characteristics between transmission and reception antennas. The number of elements of the complex transfer function is equivalent to the product of the number of transmission antennas and the number of reception antennas. In addition, PTL 3 discloses estimating the posture of a living body by using a radar cross-section (RCS) calculated from received power, with the same configuration as in PTL 2. RCS is an index indicating the area of an object that reflected a transmission wave, and the RCS of a living body changes in various ways according to the posture.

PTL 1 discloses that it is possible to know the position or state of a person that is a detection target by analyzing a component including a Doppler shift, using Fourier transform. More specifically, the temporal change of an element of a complex transfer function is recorded, and the temporal waveform thereof is Fourier-transformed. Through biological activity such as respiration or heartbeat, a living body such as a person exerts a small Doppler effect on the reflected wave. Therefore, a component including a Doppler shift includes the influence of the person. On the other hand, a component that does not include a Doppler shift is a component that is not influenced by the person, that is, a component corresponding to a reflected wave from a fixed object or a direct wave between transmission and reception antennas. Specifically, it is possible to know the position or state of a person that is a detection target, by using a component included in a predetermined frequency range in a Fourier-transformed waveform.

PTL 2 discloses a method of recording a temporal change in an element of a complex transfer function, and extracting a component including a small Doppler shift including the influence of a living body by analyzing difference information of the temporal change. Specifically, it is possible to know the position or state of a person that is a detection target by using the difference information.

In contrast, PTL 3 discloses an OFDM Doppler radar that transmits a pulse using an OFDM signal, and detects a Doppler shift caused by a traveling body that is a target. Furthermore, PTL 4 discloses, with regard to an OFDM Doppler radar, a high-speed processing method that does not require Fourier transform.

Furthermore, PTL 6 and 7 disclose techniques for improving the accuracy of estimation of complex transfer functions between transmission and reception antennas, by transmitting an OFDM signal. PTL 5 discloses that received noise components can be reduced by averaging complex transfer functions on a subcarrier basis, and PTL 7 discloses that received noise components can be reduced by selecting a subcarrier having the maximum reception power.

However, in the methods in PTL 1, 2, and 3, non-modulated waves are transmitted, and thus it is difficult to make use of commercially available devices, and dedicated hardware is required. Specifically, it is not possible to use communication devices that are currently widely used, and thus a user needs to additionally provide dedicated hardware aside from an existing communication device.

Furthermore, in order to obtain sufficient accuracy with the methods in PTL 4 and 5, it is necessary to make pulses steep, which requires a wide frequency band. As such, the cost of hardware is more expensive compared to communication devices for public use.

In the technique in NPL 1, by transmitting and receiving signals having a plurality of frequencies using a measuring device such as a network analyzer, it is possible to estimate the time of flight (ToF) and distance, which can be computed from the ToF, between a transmission antenna and a reception antenna. As in a ranging sensor that uses a frequency modulated continuous wave (FMCW) radar, this makes use of the property in which, when two signals having different frequencies are transmitted at the same phase, the phase received by the reception antenna changes depending on the frequency difference between signals and the propagation distance between the antennas. The technique in NPL 1 improves resolution by performing ToF estimation using the multiple signal classification (MUSIC) method. However, it is necessary for the transmission side and reception side to either operate with the same reference frequency or be synchronized with high accuracy, and thus the technique cannot be used in household appliances connected via, for example, a wireless LAN. Furthermore, only the distance between antennas can be estimated, and, for example, the distance to a living body that is not equipped with a special device cannot be estimated.

In view of the above, the inventors arrived at inventing, with high accuracy and low cost by using an existing transmission device, an estimating device, etc., capable of more accurately estimating the distance from the estimating device to a living body, and so on, using a multicarrier radio signal represented by an OFDM signal.

An estimating device according to a first aspect of the present disclosure is a device that estimates a distance to a living body and an angle, and includes: a transmission signal generator that generates a multicarrier signal obtained by modulating S subcarrier signals, where S is a natural number greater than or equal to 2; a transmission antenna including M transmission antenna elements, where M is a natural number greater than or equal to 1; a transmitter that causes the transmission antenna to transmit the multicarrier signal, by processing and outputting the multicarrier signal to the transmission antenna; a reception antenna including N reception antenna elements, where N is a natural number greater than or equal to 1, where at least one of M or N is greater than or equal to 2; a receiver that measures, for a first period equivalent to a cycle derived from an activity of the living body, a plurality of reception signals which are received by each of the N reception antenna elements and include a reflected signal which is the multicarrier signal transmitted from each of the M transmission antenna elements that has been reflected or dispersed by the living body; a first complex transfer function calculator that calculates, for each of S subcarriers to which the S subcarrier signals correspond, a plurality of first complex transfer functions indicating a propagation characteristic between a transmission antenna element and a reception antenna element in each of M×N combinations which are combinations of each of the M transmission antenna elements and each of the N reception antenna elements, using the plurality of reception signals measured by the receiver in the first period, and records the plurality of first complex transfer functions sequentially in time series which is an order in which the plurality of reception signals are measured; a second complex transfer function calculator that calculates a second complex transfer function by dividing all elements based on the plurality of first complex transfer functions by a direct wave component extracted using one or more elements based on the plurality of first complex transfer functions, the direct wave component having arrived not via the living body from the plurality of reception signals; a third complex transfer function calculator that calculates, from the second complex transfer function, a third complex transfer function by correcting, with respect to a reference phase calculated from a positional relationship between the M transmission antenna elements and the N reception antenna elements, an frequency phase error in the S subcarriers of the second complex transfer function, and an antenna phase error in the M transmission antenna elements; a living body correlation matrix calculator that calculates a living body correlation matrix having M×N rows and columns for each of the S subcarriers, by extracting a component related to the living body from the third complex transfer function; and an estimator that estimates, using the living body correlation matrix calculated for each of the S subcarriers, a third distance that is a sum of a first distance between the transmission antenna and the living body and a second distance between the reception antenna and the living body, and a first angle that is a direction of the living body as seen from the transmission antenna or the reception antenna.

With this configuration, a living body radar that measures distance to a living body can be realized by repurposing an existing communication device by using a multicarrier signal such as an OFDM signal as a transmission signal. For example, reception devices of multicarrier signals such as OFDM signals are already widely used as mobile phones, television broadcast reception devices, wireless LAN devices, and so on, and thus a living body radar that measures the distance to a living body can be realized at a lower cost than when non-modulated signals are used.

An estimating device according to a second aspect of the present disclosure is the estimating device according to the first aspect, wherein the third complex transfer function calculator includes a frequency phase corrector that calculates a frequency phase correction value for correcting a frequency phase error in the S subcarriers for each of M×N propagation paths between the M transmission antenna elements and the N reception antenna elements, based on (i) ideal complex transfer functions in each of the M×N propagation paths obtained based on M×N inter-antenna distances between each of the M transmission antenna elements and each of the N reception antenna elements, and (ii) a reference complex transfer function matrix including M×N complex transfer functions measured in a second period, and corrects complex transfer functions for the first period. For this reason, error due to the influence of phase characteristics of circuits or antennas inside the transmitter and receiver can be removed, and thus the distance from the estimating device to the living body can be more accurately estimated.

An estimating device according to a third aspect of the present disclosure is the estimating device according to the first or second aspect, wherein the third complex transfer function calculator includes an antenna phase corrector that calculates an antenna phase correction value for correcting an antenna phase error in the M x N combinations for each of the S subcarriers, based on (i) ideal complex transfer functions in each of M×N propagation paths between the M transmission antenna elements and the N reception antenna elements obtained based on M inter-antenna distances between the M transmission antenna elements and the N reception antenna elements, and (ii) a reference complex transfer function matrix including M×N complex transfer functions measured in a second period, and corrects complex transfer functions for the first period. For this reason, error due to the influence of phase characteristics of circuits or antennas inside the transmitter and receiver can be removed, and thus the distance from the estimating device to the living body can be more accurately estimated.

An estimating device according to a fourth aspect of the present disclosure is the estimating device according to any one of the first to third aspects, further including: a matrix transformer that transforms the third complex transfer function including M×N×S elements into a complex transfer function vector with dimensions of A×1 or 1×A, where A is an integer greater than or equal to 2 and less than or equal to M×N×S, wherein the living body correlation matrix calculator calculates the living body correlation matrix having M×N rows and columns for each of the S subcarriers based on the complex transfer function vector with dimensions of 1×A. For this reason, the position of the living body relative to the estimating device can be more accurately estimated.

An estimating device according to a fifth aspect of the present disclosure is the estimating device according to any one of the first to fourth aspects, wherein the estimator estimates the third distance and the first angle using any one of a multiple signal classification (MUSIC) method, a Capon method, or a beamformer method. For this reason, the position of the living body relative to the estimating device can be more accurately estimated.

An estimating method according to a sixth aspect of the present disclosure is a method of estimating a distance to a living body and an angle, and includes: generating a multicarrier signal obtained by modulating S subcarrier signals, where S is a natural number greater than or equal to 2; causing a transmission antenna to transmit the multicarrier signal, by processing and outputting the multicarrier signal to the transmission antenna, the transmission antenna including M transmission antenna elements, where M is a natural number greater than or equal to 1; measuring, for a first period equivalent to a cycle derived from an activity of the living body, a plurality of reception signals which are received by each of N reception antenna elements included in a reception antenna and which include a reflected signal which is the multicarrier signal transmitted from each of the M transmission antenna elements that has been reflected or dispersed by the living body, where N is a natural number greater than or equal to 1, where at least one of M or N is greater than or equal to 2; calculating, for each of S subcarriers to which the S subcarrier signals correspond, a plurality of first complex transfer functions indicating a propagation characteristic between a transmission antenna element and a reception antenna element in each of M×N combinations which are combinations of each of the M transmission antenna elements and each of the N reception antenna elements, using the plurality of reception signals measured in the first period, and recording the plurality of first complex transfer functions sequentially in time series which is an order in which the plurality of reception signals are measured; calculating a second complex transfer function by dividing all elements based on the plurality of first complex transfer functions by a direct wave component extracted using one or more elements based on the plurality of first complex transfer functions, the direct wave component having arrived not via the living body from the plurality of reception signals; calculating, from the second complex transfer function, a third complex transfer function by correcting, with respect to a reference phase calculated from a positional relationship between the M transmission antenna elements and the N reception antenna elements, a frequency phase error in the S subcarriers of the second complex transfer function, and an antenna phase error in the M transmission antenna elements; calculating a living body correlation matrix having M×N rows and columns for each of the S subcarriers, by extracting a component related to the living body from the third complex transfer function; and estimating, using the living body correlation matrix calculated for each of the S subcarriers, a third distance that is a sum of a first distance between the transmission antenna and the living body and a second distance between the reception antenna and the living body, and a first angle that is a direction of the living body as seen from the transmission antenna or the reception antenna.

With this configuration, a living body radar that measures distance to a living body can be realized by repurposing an existing communication device by using a multicarrier signal such as an OFDM signal as a transmission signal. For example, reception devices of multicarrier signals such as OFDM signals are already widely used as mobile phones, television broadcast reception devices, wireless LAN devices, and so on, and thus a living body radar that measures the distance to a living body can be realized at a lower cost than when non-modulated signals are used.

A program according to a seventh aspect of the present disclosure is a program for causing a computer to execute the estimating method according to the sixth aspect.

It should be noted that these generic and specific aspects may be implemented using a system, an integrated circuit, a computer program, or a computer-readable recording medium such as a CD-ROM, or any combination of an apparatus, a system, a method, an integrated circuit, a computer program, or a recording medium.

Hereinafter, exemplary embodiments of the present disclosure will be described in detail with reference to the Drawings. It should be noted that each of the exemplary embodiments described hereinafter illustrate a specific example of the present disclosure. The numerical values, shapes, materials, elements, the arrangement and connection of the elements, steps, the processing order of the steps, etc., shown in the following exemplary embodiments are mere examples, and are therefore not intended to limit the present disclosure. Furthermore, among elements in the following exemplary embodiments, those not recited in any one of the independent claims defining the most generic concept of the present disclosure are described as optional elements making up a more preferable form. It should be noted that in the Specification and the Drawings, elements having substantially the same functional configuration are given the same numerical sign in order to omit overlapping descriptions.

### Embodiment 1

Hereinafter, a method with which the distance and direction to a living body that is a detection target from estimating device 100 is estimated by estimating device 100 according to Embodiment 1 will be described with reference to the drawings.

In Embodiment 1, an example in which the MISO scheme in which the transmission antenna is plural in number will be given. It should be noted that the present disclosure can similarly be applied to a single input multiple output (SIMO) scheme in which there are a plurality of reception antennas or a multiple input multiple output (MIMO) scheme in which there are a plurality of both transmission antennas and reception antennas.

### [Configuration of Estimating Device 100]

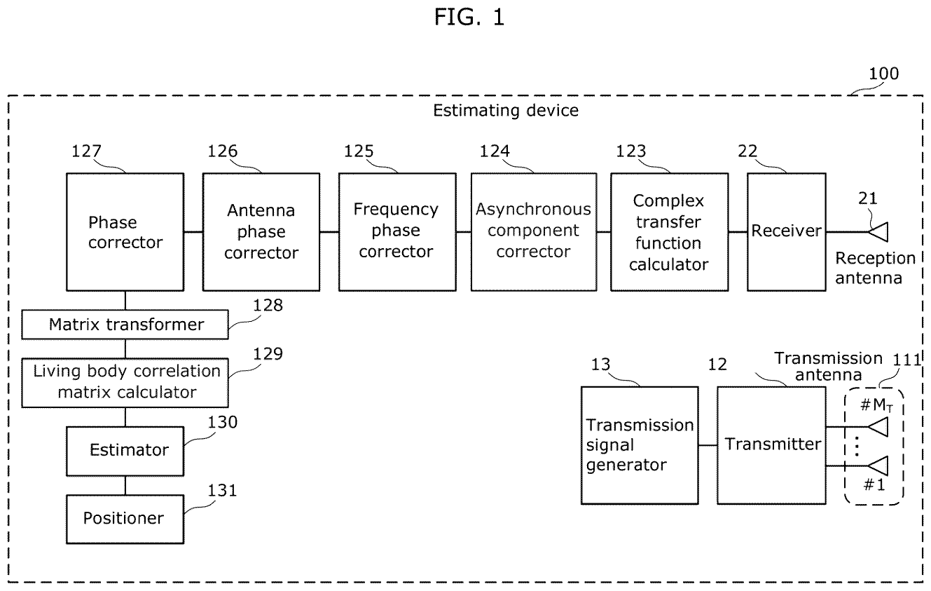

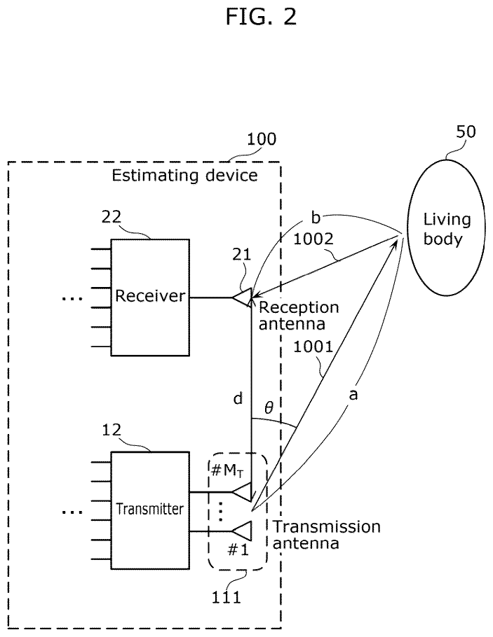

FIG. 1 is a block diagram illustrating an example of a configuration of estimating device 100 according to Embodiment 1. FIG. 2 is a diagram illustrating an example of a detection target of estimating device 100 illustrated in FIG. 1.

Estimating device 100 illustrated in FIG. 1 includes transmission antenna 111, transmitter 12, transmission signal generator 13, reception antenna 21, receiver 22, complex transfer function calculator 123, asynchronous component corrector 124, frequency phase corrector 125, antenna phase corrector 126, phase corrector 127, matrix transformer 128, living body correlation matrix calculator 129, estimator 130, and positioner 131. Estimating device 100 estimates the position of living body 50 using estimating device 100 as a directional or positional reference.

### [Transmission Antenna 111]

Transmission antenna 111 includes M (M is a natural number of at least 2) transmission antenna elements. As described above, the transmission antenna element transmits a multicarrier signal (transmission wave) generated by transmitter 12 to be described later.

### [Transmission Signal Generator 13]

Transmission signal generator 13 generates a multicarrier signal obtained by modulating S subcarrier signals for each of the M transmission antenna elements included in transmission antenna 111. Transmission signal generator 13 generates S subcarrier signals corresponding to S subcarriers having mutually different frequency bands, and generates a multicarrier signal by multiplexing the generated S subcarrier signals. In the present embodiment, transmission signal generator 13 is exemplified as generating an OFDM signal as a multicarrier signal. However, aside from generating an OFDM signal in which respective subcarriers are orthogonal, other multicarrier signals such as a simple frequency division multiplexing (FDM) signal may be generated as long as it is a multicarrier signal obtainable by multicarrier modulation. It should be noted that an OFDM signal has a high frequency band utilization efficiency, and is, for example, a signal in which S subcarrier signals corresponding to S subcarriers are multiplexed.

Furthermore, the signal generated by transmission signal generator 13 may be a signal that is shared with a signal used for communication.

### [Transmitter 12]

Transmitter 12 adds appropriate processing to the signal generated by transmission signal generator 13, to generate a transmission wave. The processing carried out here includes, for example, up-conversion in which the signal is converted from the intermediate frequency (IF) frequency band to the radio frequency (RF) frequency band, amplification in which the signal is amplified to the appropriate transmission level, etc. Then, as illustrated in FIG. 2, transmitter 12 outputs the processed multicarrier signal to transmission antenna 111 to thereby cause transmission antenna 111 to transmit the multicarrier signal. With this, the multicarrier signal is transmitted from the M (M is a natural number greater than or equal to 2) transmission antenna elements included in transmission antenna 111.

### [Reception Antenna 21]

Reception antenna 21 includes N reception antenna elements. Here, N is a natural number greater than or equal to 1. In the present embodiment, reception antenna 21 includes one reception antenna element. Then, for example, as illustrated in FIG. 2, the one reception antenna element receives a signal that was transmitted by the M transmission antenna elements and reflected by living body 50 (i.e., a reception signal).

### [Receiver 22]

Receiver 22 measures, for a first period equivalent to a cycle derived from an activity of living body 50, the reception signals that are received by the single reception antenna element and include reflected signals which are the multicarrier signals transmitted from the M transmission antenna elements that have been reflected or dispersed by living body 50. A cycle derived from the activity of the living body is a living body-derived cycle (living body fluctuation cycle) which is a time period greater than or equal to a half-cycle of any of the cycles of respiration, heartbeat, and body motion of living body 50.

Receiver 22 converts the high-frequency signal received by the single reception antenna element into a low-frequency signal on which signal processing can be performed. Then, receiver 22 demodulates the M OFDM signals transmitted by the M transmission antenna elements into S×M subcarrier signals. Each of the S×M subcarrier signals is represented by an IQ symbol. Receiver 22 outputs, to complex transfer function calculator 123, the S×M sets of subcarrier signals obtained by converting the high-frequency signal received by the N (one in the present embodiment) reception antenna elements, for at least the first period.

It should be noted that, receiver 22 may continue to measure the reception signals already received by reception antenna 21, and continuously or periodically transmit the S×M subcarrier signals (IQ symbols) to complex transfer function calculator 123.

### [Complex Transfer Function Calculator 123]

Complex transfer function calculator 123, using the reception signals measured in the first period by receiver 22, calculates, for each of the S subcarriers to which the S subcarrier signals correspond, a plurality of complex transfer functions indicating propagation characteristics between a transmission antenna element and a reception antenna element in each of M×N combinations (M combinations in the present embodiment) which are the combinations of each of the M (two or more in the present embodiment) transmission antenna elements and each of the N (one in the present embodiment) reception antenna elements. It should be noted that the M×N combinations are all the obtainable one-to-one combinations between the M transmission antenna elements and the N reception antenna elements.

In the present embodiment, complex transfer function calculator 123 calculates, using the S×M subcarrier signals transmitted from receiver 22, first complex transfer functions indicating the propagation characteristics between each of the transmission antenna elements and each of the reception antenna elements, for each of the S×M subcarrier signals.

It should be noted that the calculated first complex transfer function matrix also includes reflected waves that did not arrive via living body 50, such as direct waves and reflected waves derived from a fixed object.

The method of calculating a first complex transfer function from one subcarrier signal includes, for example, a method of dividing a reception IQ symbol by a known signal such as a pilot signal or a guard interval signal.

It should be noted that complex transfer function calculator 123 may constantly calculate the complex transfer function matrix using each of the S subcarrier signals outputted continuously or on a regular basis by receiver 22. By adopting this configuration, when estimating device 100 shares the hardware of a communication device, the complex transfer function matrix that is normally calculated for use in processing by the communication device can also be used by estimating device 100. Complex transfer function calculator 123 is one example of a first complex transfer function calculator.

It should be noted that complex transfer function calculator 123 may perform singular value decomposition on the first complex transfer function according to equation 1, and output the right singular vector V obtained by the singular value decomposition instead of the first complex transfer function ho.

[Math. 1]

h₀=UΣV^{H} (Equation 1)

Here, H represents a Hermitian matrix. As a result, the amount of data to be transmitted downstream can be reduced. In the case of performing singular value decomposition, the same processing is possible by replacing h with V in the subsequent explanations.

### [Asynchronous Component Corrector 124]

The first complex transfer function matrix calculated in complex transfer function calculator 123 includes an error component of a phase that varies with time. This error component is called an asynchronous component. The asynchronous component occurs due to clock fluctuations between transmitter 12 and receiver 22, timing fluctuations in digital-to-analog conversion of the transmission signal or analog-to-digital conversion of the reception signal, and the like.

Asynchronous component corrector 124 calculates a second complex transfer function by removing the asynchronous component from the first complex transfer function matrix while preserving the phase change derived from living body 50.

In the present embodiment, a method using singular value decomposition as a method for removing the asynchronous component will be described. The asynchronous component is equally superimposed on components that have passed through any propagation path between transmission antenna 111 and reception antenna 21. In contrast, the phase change derived from living body 50 is superimposed only on components that have passed through propagation paths reflected and scattered by living body 50. Specifically, asynchronous component corrector 124 extracts components with little influence of reflection or scattering by living body 50 from the first complex transfer function, and divides the entire function by these components. Thereby, asynchronous component corrector 124 can remove the asynchronous component from the first complex transfer function while preserving the component derived from living body 50. More specifically, when the first complex transfer function matrix is h₀(t), asynchronous component corrector 124 performs singular value decomposition into left singular vector U (t), right singular vector V (t), and singular value vector Σ(t) according to Equation 2.

$$[ Math . 2 ]$$

$${h}_{0} ( t ) = U ( t ) \sum ( t ) {V}^{H} ( t ) \qquad ( Equation 2 )$$

$$U ( t ) = [ \begin{matrix}{u}_{1} ( t ) & {u}_{2} ( t ) & ⋯ & {u}_{M} ( t )\end{matrix} ] \qquad ( Equation 2 - 1 )$$

$$V ( t ) = [ \begin{matrix}{v}_{1} ( t ) & {v}_{2} ( t ) & ⋯ & {v}_{S} ( t )\end{matrix} ] \qquad ( Equation 2 - 2 )$$

The singular value vectors obtained in this manner represent the respective propagation paths of a plurality of transmission signals between transmission antenna 111 and reception antenna 21, and left singular vector u₁(t) and right singular vector v₁(t) corresponding to the maximum singular value correspond to the direct wave propagation between transmission antenna 111 and reception antenna 21.

Next, asynchronous component corrector 124 calculates a second complex transfer function matrix h′(t) with corrected asynchronous components using left singular vector u₁(t) and right singular vector v₁(t) as shown in Equation 3.

$$[ Math . 3 ]$$

$${h}^{′} ( t ) = {h}_{0} ( t ) / ( {u}_{1}^{H} {h}_{0} ( t ) {v}_{1} ) \qquad ( Equation 3 )$$

In the present embodiment, a method using singular value decomposition has been described, but among the elements of the complex transfer function matrix having M rows and S columns, an element that will serve as a reference may be determined, and the complex transfer function matrix at each time point may be normalized (divided) by the element serving as the reference. Even with this operation, it is possible to remove the asynchronous component. It should be noted that the element of the complex transfer function that will serve as a reference is one example of a direct wave component that did not arrive via living body 50, extracted from a plurality of reception signals. It should be noted that the element of the complex transfer function that will serve as a reference is not limited to an element obtained by singular value decomposition, but may be any one of the elements of the first complex transfer function, may be the average of a plurality of elements of the first complex transfer function, or may be a direct wave component obtained by Eigendecomposition of a correlation matrix of the first complex transfer function.

In this manner, asynchronous component corrector 124 calculates a second complex transfer function by dividing all elements of the first complex transfer function by a direct wave component extracted using one or more elements of the first complex transfer function. A direct wave component is a component that did not arrive via living body 50, extracted from a plurality of reception signals. Asynchronous component corrector 124 is one example of a second complex transfer function calculator.

### [Frequency Phase Corrector 125]

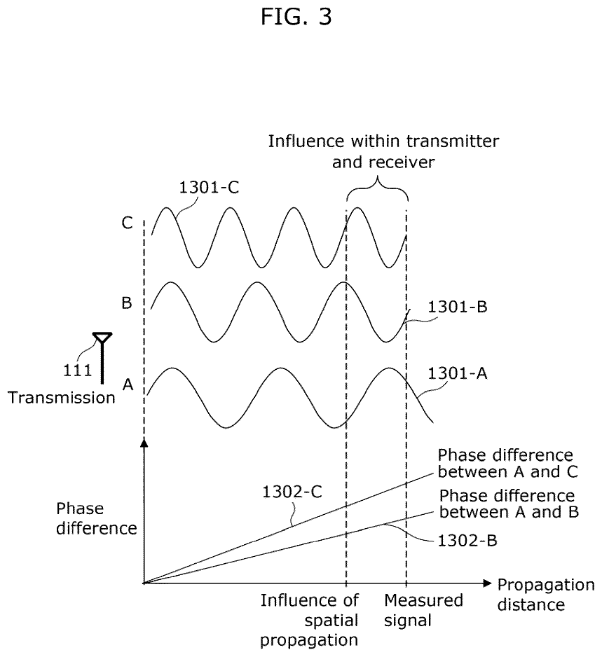

Frequency phase corrector 125 obtains the second complex transfer function matrix h′ calculated by asynchronous component corrector 124, and calculates the frequency phase correction value h_{cal1} for correcting the phase error in the frequency direction. The phase error in the frequency direction is a phase error between a plurality of signals having mutually different frequencies. Phase errors that require correction will be described with reference to FIG. 3. FIG. 3 is a schematic diagram illustrating that the phase of a reception signal changes due to frequency and distance.

When signals of different frequencies propagate through a space and are received, the amount of phase rotation by a transmission signal with respect to the reception signal is different depending on the frequency and the distance between the transmission antenna and the reception antenna (hereafter referred to as inter-antenna distance). FIG. 3 illustrates three transmission waves 1301-A, 1301-B, and 1301-C, which are signals with mutually different frequencies transmitted from transmission antenna 111 with the same phase, and it can be seen that the phases continue to differ as the propagation distance increases (1302-B, 1302-C). For this reason, the inter-antenna distance can be calculated by transmitting and receiving signals having a plurality of already-known frequencies, measuring phase differences, and performing back calculation. However, the phase difference that is actually measured includes, not only the influence of spatial propagation between the transmission antenna and the reception antenna, but also error due to the influence of phase characteristics of internal circuits and antennas of the transmission device and reception device (hereinafter such an error is referred to as a phase error). For this reason, in order to correctly measure the inter-antenna distance, it is necessary to remove the phase error from the measured signal.

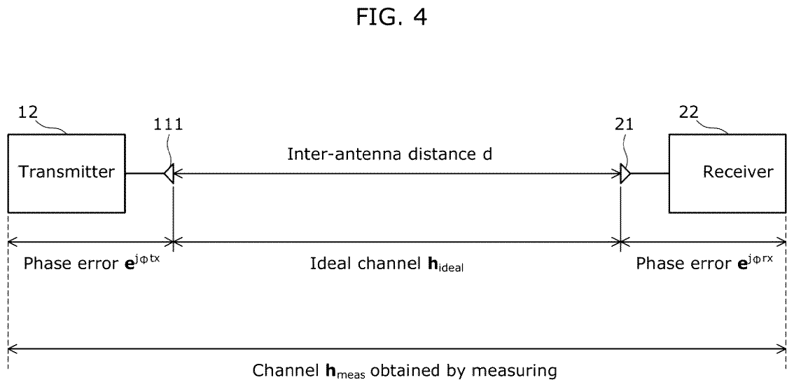

FIG. 4 is a diagram illustrating the correspondence between the previously described phase error and a channel (complex transfer function).

The phase error can be calculated by calculating the difference between channel hₘₑₐₛ indicated by a matrix obtained by measurement and an ideal channel h_{ideal} of the space indicated by a matrix that can be calculated from the inter-antenna distance. This is not limited to estimating the inter-antenna distance but also applies to the case of estimating the distance to living body 50.

Next, the specific operation of frequency phase corrector 125 will be described. Frequency phase corrector 125 obtains the second complex transfer function matrix h′, and corrects the phase error in the frequency direction. Here, a frequency phase error refers to, among differences with respect to the phase of reference subcarrier signal SO in the second complex transfer function matrix, a phase difference not resulting from spatial propagation between the antennas. More specifically, the frequency phase error includes errors due to the influence of the frequency characteristics of transmission antenna 111 and reception antenna 21, the electrical length of the internal circuitry of transmitter 12, the electrical length of the internal circuitry of receiver 22, and so on. The phase error includes phase difference e^{jφtx} caused by transmission antenna 111 and transmitter 12 and phase error e^{jφrx} caused by reception antenna 21 and receiver 22.

The second complex transfer function matrix h′ received by frequency phase corrector 125 is expressed using the following Equation 4.

$$\begin{matrix}[ Math . 4 ] & \\ {h}^{′} = ( \begin{matrix}{h}_{11} & … & {h}_{1 S} \\ ⋮ & ⋱ & ⋮ \\ {h}_{M 1} & … & {h}_{MS}\end{matrix} ) & ( Equation 4 )\end{matrix}$$

The second complex transfer function matrix h′ is an M×S matrix. The M elements included in the same column of the second complex transfer function matrix h′ are elements generated by reception signals of the same subcarrier (that is, the same frequency), and are elements based on signals transmitted by different M transmission antenna elements. The S elements included in the same row of the second complex transfer function matrix h′ are elements generated by signals of the same transmission antenna element, and are elements based on different S subcarrier signals. Frequency phase corrector 125 calculates the frequency phase correction value for each row of the second complex transfer function matrix, that is, for each antenna, using a predetermined method. Here, the method of calculating the correction value will be described focusing on the j-th row. First, frequency phase corrector 125 calculates h_{ideal1}, which is the ideal inter-antenna element channel, based on distance dⱼ between the j-th transmission antenna element and reception antenna element that is inputted in advance. Here, h_{ideal1} is a vector represented by the complex number, and has S elements which is the subcarrier number. The i-th element is calculated by Equation 5.

$$\begin{matrix}[ Math . 5 ] & \\ {h}_{ideal 1 ( i )} = exp ( - {jk}_{i} {d}_{j} ) & ( Equation 5 )\end{matrix}$$

Here, kᵢ is the wavenumber of the i-th subcarrier. In this manner, h_{ideal1} is an ideal complex transfer function between the transmission antenna element and the reception antenna element obtainable based on the inter-antenna distance between the transmission antenna element and the reception antenna element.

Next, frequency phase corrector 125 obtains, from asynchronous component corrector 124, a reference complex transfer function matrix that includes M×S complex transfer function matrices measured in a second period. The second period is equivalent to a cycle derived from an activity of living body 50. A cycle derived from the activity of the living body is a living body-derived cycle (living body fluctuation cycle) which is a time period greater than or equal to a half-cycle of any of the cycles of respiration, heartbeat, and body motion of living body 50. It should be noted that measurement of the reference complex transfer function matrix is preferably performed in a person-free state in which there is little influence from a living body, but may include the influence of a living body. An initial complex transfer function matrix obtained from complex transfer function calculator 123 may be used for the reference complex transfer function matrix. Frequency phase corrector 125 may calculate a new reference complex transfer function matrix based on data of a timing at which fluctuation obtained by simultaneously calculating the temporal fluctuations of absolute values of complex transfer functions is small, and update the reference complex transfer function matrix with the new reference complex transfer function matrix calculated. In the present embodiment, since there are M transmission antenna elements and one reception antenna element, the reference complex transfer function matrix is the matrix hₘₑₐₛ having an element number of S×M.

Next, frequency phase corrector 125 calculates the frequency phase correction value h_{cal1} for correcting the frequency phase errors in S subcarriers, based on the ideal channel h_{ideal1} and the reference complex transfer function (channel hₘₑₐₛ). More specifically, frequency phase corrector 125 calculates the ratio between h_{ideal1}, which is the ideal channel obtained by calculation, and the measured reference complex transfer function matrix hₘₑₐₛ, and sets the ratio as the frequency phase correction value h_{cal1}. Specifically, the frequency phase correction value h_{cal1} is calculated using the following Equation 6.

$$\begin{matrix}[ Math . 6 ] & \\ {h}_{cal 1} = {h}_{ideal 1} \text{?} {h}_{meas} & ( Equation 6 )\end{matrix}$$

Here, Ø represents Hadamard division, which is element-wise division of vectors.

The frequency phase correction value h_{cal1} is the same as long as the reference complex transfer function matrix does not change.

For this reason, frequency phase corrector 125 may store the calculated frequency phase correction value h_{cal1} in a memory or the like, and use the frequency phase correction value h_{cal1} stored in the memory or the like from the next time onward. Stated differently, once frequency phase corrector 125 calculates the frequency phase correction value h_{cal1}, it does not need to calculate the frequency phase correction value h_{cal1} from the next time onward.

### [Antenna Phase Corrector 126]

Antenna phase corrector 126 obtains the second complex transfer function matrix h′ calculated by asynchronous component corrector 124, and calculates antenna phase correction value h_{cal2} for correcting the antenna phase error. Here, an antenna phase error refers to, among differences with respect to the phase of the reference transmission antenna element in the second complex transfer function matrix h′, a phase difference not resulting from spatial propagation between the antennas. More specifically, the antenna phase error includes errors due to the influence of the frequency characteristics of transmission antenna 111 and reception antenna 21, the electrical length of the internal circuitry of transmitter 12, the electrical length of the internal circuitry of receiver 22, and so on. The antenna phase error includes phase error e^{jφtx} caused by transmission antenna 111 and transmitter 12 and phase error e^{jφrx} caused by reception antenna 21 and receiver 22.

The second complex transfer function matrix h′ received by antenna phase corrector 126 is expressed using the following Equation 7.

$$\begin{matrix}[ Math . 7 ] & \\ {h}^{′} = ( \begin{matrix}{h}_{11} & … & {h}_{1 S} \\ ⋮ & ⋱ & ⋮ \\ {h}_{M 1} & … & {h}_{MS}\end{matrix} ) & ( Equation 7 )\end{matrix}$$

The second complex transfer function matrix h′ is an M×S matrix. The M elements included in the same column of the second complex transfer function matrix h′ are elements generated by reception signals of the same subcarrier (that is, the same frequency), and are elements based on signals transmitted by different M transmission antenna elements. The S elements included in the same row of the second complex transfer function matrix h′ are elements generated by signals of the same transmission antenna element, and are elements based on different S subcarrier signals. Antenna phase corrector 126 calculates the antenna phase correction value for each column of the second complex transfer function matrix, that is, for each frequency, using a predetermined method. Here, the method of calculating the correction value will be described focusing on the i-th column. First, antenna phase corrector 126 calculates h_{ideal2}, which is the ideal inter-antenna element channel, based on distance dⱼ between the j-th transmission antenna element and reception antenna element that is inputted in advance. Here, h_{ideal2} is a vector represented by the complex number, and has M elements which is the transmission antenna element number. The j-th element is calculated by Equation 8.

$$\begin{matrix}[ Math . 8 ] & \\ {h}_{ideal 2 ( j )} = exp ( - {jk}_{i} {d}_{j} ) & ( Equation 8 )\end{matrix}$$

Here, kᵢ is the wavenumber of the i-th subcarrier. In this manner, h_{ideal2} is an ideal complex transfer function between the transmission antenna element and the reception antenna element obtainable based on the inter-antenna distance between the transmission antenna element and the reception antenna element.

Next, antenna phase corrector 126 obtains, from asynchronous component corrector 124, a reference complex transfer function matrix that includes M×S complex transfer function matrices measured in a second period. The second period is equivalent to a cycle derived from an activity of living body 50. A cycle derived from the activity of the living body is a living body-derived cycle (living body fluctuation cycle) which is a time period greater than or equal to a half-cycle of any of the cycles of respiration, heartbeat, and body motion of living body 50. It should be noted that measurement of the reference complex transfer function matrix is preferably performed in a person-free state in which there is little influence from a living body, but may include the influence of a living body. An initial second complex transfer function matrix obtained from asynchronous component corrector 124 may be used for the reference complex transfer function matrix. Antenna phase corrector 126 may calculate a new reference complex transfer function matrix based on data of a timing at which fluctuation obtained by simultaneously calculating the temporal fluctuations of absolute values of complex transfer functions is small, and update the reference complex transfer function matrix with the new reference complex transfer function matrix calculated. A reflected wave from a living body at a position that is known in advance may be used as the reference complex transfer function matrix. In the present embodiment, since there are M transmission antenna elements and one reception antenna element, the reference complex transfer function matrix is the matrix hₘₑₐₛ having an element number of S×M.

Next, antenna phase corrector 126 calculates the correction value for correcting the antenna phase errors in the M transmission antenna elements, based on the ideal channel h_{ideal2} and the reference complex transfer function (channel hₘₑₐₛ). More specifically, antenna phase corrector 126 calculates the ratio between h_{ideal2}, which is the ideal channel obtained by calculation, and the measured reference complex transfer function matrix hₘₑₐₛ, and sets the ratio as the antenna phase correction value h_{cal2}. Specifically, the antenna phase correction value h_{cal2} is calculated through the following calculation.

$$\begin{matrix}[ Math . 9 ] & \\ {h}_{cal 2} = {h}_{ideal 2} \text{?} {h}_{meas} & ( Equation 9 )\end{matrix}$$

Here, Ø represents Hadamard division, which is element-wise division of vectors.

The antenna phase correction value h_{cal2} is the same as long as the reference complex transfer function matrix does not change. For this reason, antenna phase corrector 126 may store the calculated antenna phase correction value h_{cal2} in a memory or the like, and use the frequency value stored in the memory or the like from the next time onward. Stated differently, once antenna phase corrector 126 calculates the antenna phase correction value h_{cal2}, it does not need to calculate the antenna phase correction value h_{cal2} from the next time onward.

It should be noted that although the operation of frequency phase corrector 125 was described before the operation of antenna phase corrector 126 in the present example, the actual execution order is not fixed. Stated differently, the operation of frequency phase corrector 125 may be performed before, after, or simultaneously with (in parallel with) the operation of antenna phase corrector 126.

### [Phase Corrector 127]

Next, based on the frequency phase correction value h_{cal1} and the antenna phase correction value h_{cal2}, phase corrector 127 corrects the second complex transfer function matrix h′ according to the following Equation 10, and calculates a third complex transfer function matrix h″.

$$\begin{matrix}[ Math . 10 ] & \\ {h}^{″} = ∠ {h}_{cal 1} ∘ ∠ {h}_{cal 2} ∘ {h}^{′} & ( Equation 10 )\end{matrix}$$

Here, ∠h_{cal} is a matrix representing the phase angle, which is obtained by normalizing the absolute value of each element of h_{cal}, which is a complex number, to 1. ∘ represents Hadamard product, which is element-wise multiplication.

It should be noted that since the second complex transfer function matrix h′ is corrected by frequency phase corrector 125, antenna phase corrector 126, and phase corrector 127 and the third complex transfer function matrix h″ is calculated, frequency phase corrector 125, antenna phase corrector 126, and phase corrector 127 are one example of a third complex transfer function calculator that calculates the third complex transfer function matrix h″. The third complex transfer function calculator calculates a third complex transfer function matrix h″ by correcting the second complex transfer function matrix h′ using offset values with respect to a reference phase calculated from the positional relationship between the M transmission antenna elements and the reception antenna element, where these offset values correct for frequency phase errors in the S subcarriers and antenna phase errors in the M transmission antenna elements of the second complex transfer function matrix h′.

### [Matrix Transformer 128]

The third complex transfer function matrix h″ calculated in phase corrector 127 is expressed using Equation 11.

$$\begin{matrix}[ Math . 11 ] & \\ {h}^{″} = ( \begin{matrix}{h}_{11} & … & {h}_{1 S} \\ ⋮ & ⋱ & ⋮ \\ {h}_{M 1} & … & {h}_{MS}\end{matrix} ) & ( Equation 11 )\end{matrix}$$

Matrix transformer 128 extracts any one row and one column from the third complex matrix, rearranges the extracted one row and one column as vectors, and calculates a first complex transfer function vector hᵥ. For example, hᵥ when the first row and the first column are extracted is represented by Equation 12.

$$\begin{matrix}[ Math . 12 ] & \\ {h}_{v} = [ {h}_{11} … {h}_{1 S} {h}_{21} … {h}_{M 1} ] & ( Equation 12 )\end{matrix}$$

### [Living Body Correlation Matrix Calculator 129]

For each of the S subcarriers and each of the M×N combinations, living body correlation matrix calculator 129 successively records, in the time-series order in which they are measured, the first complex transfer function vector hᵥ calculated by matrix transformer 128. Living body correlation matrix calculator 129 extracts, for each of the S subcarriers and each of the M×N combinations, components of the living body from the first complex transfer function vector hᵥ recorded in time series and measured for the first period, to thereby calculate, for each of the S subcarriers, a living body component transfer function matrix expressed by a M×N-dimension matrix.

Here, the living body component transfer function matrix is the extracted reflected wave or dispersed wave (living body component) included in the reception signal that passed via living body 50. The methods of calculating the living body component from the complex transfer functions recorded in time-series include the method using Fourier transform disclosed in PTL 1 and the method using difference information disclosed in PTL 2.

For example, with the method that uses Fourier transform, by performing Fourier transform on first complex transfer function vector hᵥ for the measurement time (slow time) and extracting only specific frequency components, living body component complex transfer function vector h_{fft} can be calculated. Here, living body component complex transfer function vector heft is calculated for each of frequency components included in the frequencies, for example, 0.1 Hz to 3 Hz, which can include the influence of activities of the living body. In this method, by further performing inverse Fourier transform in the subcarrier direction on the calculated living body component complex transfer function vector het and calculating the living body component complex transfer function vector h_{ifft} expressed in the time domain, the time from when a signal including a living body component is transmitted from transmitter 12 to when it is received by receiver 22 is calculated.

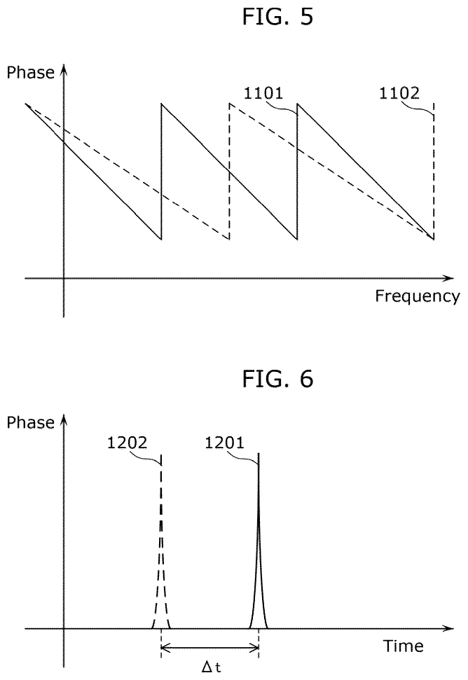

Here, the relationship between frequency (column direction of the matrix) and phase of complex transfer function vector h_{fft} is illustrated in FIG. 5. Solid line 1101 represents the fluctuation of the phase of each component of the living body component transfer function matrix according to the subcarrier frequency when living body 50 is present at a certain position. The phase here is the difference from the phase in the frequency of subcarrier SO serving as a reference. Since the length of the path of the radio wave reflected by living body 50 becomes shorter when living body 50 approaches the transmission antenna or reception antenna from the aforementioned position, the slope on the graph becomes gentle and becomes like broken line 1102. In principle, time of flight (ToF) or the distance to the living body can be estimated from this graph slope. Specifically, when time domain living body component transfer function matrix h_{ifft} is calculated by further performing inverse Fourier transform in the subcarrier direction on living body component transfer function matrix h_{fft}, the time from when a signal including a living body component is transmitted from transmitter 12 to when the signal is received by receiver 22 is obtained.

FIG. 6 illustrates the relationship between the time (column direction of the matrix) and the phase of time domain living body component transfer function matrix h_{ifft}. The phase changes of solid line 1101 and broken line 1102 in FIG. 5 appear as the peaks shown by solid line 1201 and broken line 1202, respectively. However, temporal resolution Δt of time that is calculated here and subcarrier bandwidth B are expressed by Equation 13.

$$\begin{matrix}[ Math . 13 ] & \\ Δ t = \frac{1}{B} [ s ] & ( Equation 13 )\end{matrix}$$

For example, when the bandwidth is 20 MHz, the temporal resolution is equivalent to 0.05 μs or approximately 15 m when converted to distance resolution, which does not stand up to practical use.

In view of this, in the present embodiment, resolution is improved by using the multiple signal classification (MUSIC) method.

In order to use the MUSIC method, living body correlation matrix calculator 129 calculates correlation matrix R′f (living body correlation matrix) of living body component complex transfer function vector haft according to the following Equation 14.

$$\begin{matrix}[ Math . 14 ] & \\ {R}_{f}^{′} = E [ {h}_{fft ( m )} {h}_{fft ( m )}^{H} ] & ( Equation 14 )\end{matrix}$$

Here, E[•] in Equation 14 indicates the average arithmetic processing in the frequency direction that may include the influence of the living body, in the column direction, that is, for each transmission antenna, and m indicates the index number from 1 to M of the transmission antennas.

In this manner, living body correlation matrix calculator 129 extracts components related to the living body from the third complex transfer function matrix h″, to thereby calculate a living body correlation matrix having M×N rows and columns for each of the S subcarriers.

### [Estimator 130]

Estimator 130 performs ranging and angle estimation according to the MUSIC method, using correlation matrix R′_{f} calculated by living body correlation matrix calculator 129. First, estimator 130 performs Eigendecomposition of living body correlation matrix R′_{f}, and calculates vector U_{S}′ corresponding to a signal and eigenvector U_{N}′ corresponding to noise. Here, eigenvectors corresponding to a signal are the vectors counted in order from a first eigenvector to the number of targets to be ranged, and is, for example, only the first eigenvector when the target is one person. Furthermore, when the targets are k persons (k being a natural number greater than or equal to 2), the eigenvectors corresponding to a signal are the k eigenvectors from the first eigenvector to the k-th eigenvector. In addition, eigenvectors corresponding to noise refers to eigenvectors other than the eigenvectors corresponding to a signal.

MUSIC spectrum P_{MUSIC}(I, θ) is calculated according to the following equation, using the eigenvectors obtained in the above-described manner.

$$\begin{matrix}[ Math . 15 ] & \\ {P}_{MUSIC} ( l , θ ) = \frac{{a}^{H} ( l , θ ) a ( l , θ )}{{a}^{H} ( l , θ ) {U}_{N} {U}_{N}^{H} a ( l , θ )} & ( Equation 15 )\end{matrix}$$

Here, a(I,θ) represents a steering vector, and is calculated by Equation 16.

$$\begin{matrix}[ Math . 16 ] & \\ a ( l , θ ) = {[ {e}^{- j \frac{2 π}{{λ}_{1}} ( l + {d}_{1} )} , … , {e}^{- j \frac{2 π}{{λ}_{S}} ( l + {d}_{M} )} ]}^{T} & ( Equation 16 )\end{matrix}$$

Here, the order of elements of the steering vector is the same as the transformation performed by matrix transformer 128 when calculating the first complex transfer function vector from the third complex transfer function matrix. λᵢ represents the wavelength of the i-th subcarrier, and l represents the total distance from a reference position of the transmission antenna (for example, the center of the first antenna element) via living body 50 to the reception antenna. dᵢ represents the difference between the total distance from the i-th transmission antenna element via living body 50 to the reception antenna element and the total distance from the reference transmission antenna element via living body 50 to the reception antenna element. l, which takes the maximum value of MUSIC spectrum P_{MUSIC}(I,θ) obtained in the above manner, corresponds to the sum (third distance) of distance a (first distance) and distance b (second distance) in FIG. 2. Distance a (first distance) is the distance between the transmission antenna element and living body 50. Distance b (second distance) is the distance between the reception antenna element and living body 50. θ which takes the maximum value of P_{MUSIC}(I,θ) corresponds to the estimated value of the direction (first angle) from the transmission antenna element to living body 50 in FIG. 2. Stated differently, estimator 130 can calculate the third distance by calculating I which takes the maximum value of MUSIC spectrum P_{MUSIC}(I,θ), and can estimate the first angle by calculating θ which takes the maximum value of MUSIC spectrum P_{MUSIC}(I,θ). In this manner, estimator 130 estimates the third distance that is the sum of the first distance between transmission antenna 111 and living body 50 and the second distance, and the first angle that is the direction of living body 50 as seen from the transmission antenna element, by using the living body correlation matrix calculated for each of the S subcarriers.

It should be noted that although the angle from a transmission antenna element is estimated in the present embodiment, a device including a plurality of reception antenna elements may be used and the angle from a reception antenna element may be estimated.

### [Positioner 131]

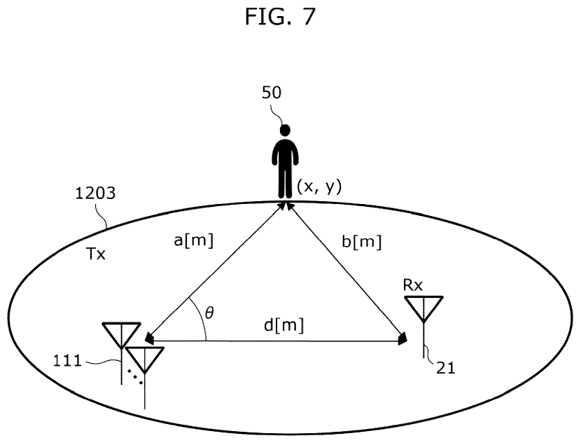

Positioner 131 calculates the coordinates of living body 50 based on third distance L and first angle θ estimated by estimator 130. FIG. 7 illustrates the relationship between living body 50, transmission antenna 111, reception antenna 21, third distance L, and first angle θ. When third distance L equivalent to the sum of first distance a and second distance b in FIG. 7 is defined, it is understood that the position of living body 50 is on the circumference of ellipse 1203, and, since first angle θ which is the angle from transmission antenna 111 is also defined, the position of living body 50 is determined to be at one point on the circumference of ellipse 1203. Hereinafter, the method for calculating the coordinates of living body 50 will be described using mathematical expressions.

First, positioner 131 calculates first distance a based on the law of cosines, using third distance L, first angle θ, and inter-antenna distance d. More specifically, first distance a is calculated using the following Equation 17.

$$\begin{matrix}[ Math . 17 ] & \\ {( L - a )}^{2} = {a}^{2} + {d}^{2} = 2 ad cos θ & ( Equation 17 )\end{matrix}$$

$$a ( 2 d cos θ - 2 L ) = {d}^{2} - {L}^{2}$$

$$∴ a = \frac{{L}^{2} - {d}^{2}}{2 ( L - d cos θ )}$$

Lastly, positioner 131 calculates the coordinates (x, y) of living body 50 using first distance a and first angle θ according to the following Equation 18.

$$\begin{matrix}[ Math . 18 ] & \\ ( x , y ) = ( a cos θ , a sin θ ) & ( Equation 18 )\end{matrix}$$

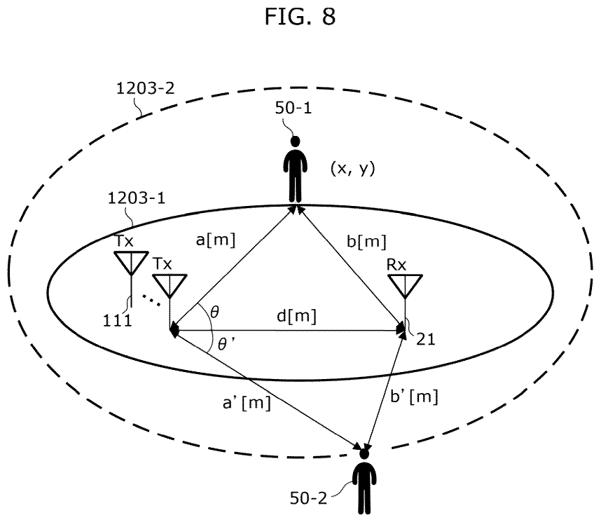

It should be noted that although in the present embodiment, matrix transformer 128 performs transformation to simultaneously include elements in the row direction and column direction of the third complex transfer function matrix, estimator 130 may perform the MUSIC method individually for each of the row direction and column direction. In this way, estimator 130 may calculate the third distance and the first angle individually. In such cases, even when the number of living bodies is two or more, estimator 130, as illustrated in FIG. 8, can estimate the third distance and the first angle for a plurality of living bodies 50-1, 50-2, then estimate the combination of the third distance and the first angle from the magnitude correlation of the Eigenvalues of the correlation matrix, and calculate the coordinates of each living body. As in the present embodiment, by expanding the steering vector to be used during MUSIC spectrum calculation into the two dimensions of distance and angle, the combinations of third distances and first angles of a plurality of persons can be estimated simultaneously. It should be noted that estimator 130 can estimate the angle or the distance from a different transmission or reception antenna element at the same time by further expanding the dimensions of the steering vector.

When estimating the third distance and the first angle individually, estimator 130 can improve the estimation accuracy of the third distance or the first angle by performing singular value decomposition or Eigendecomposition on the third complex transfer function matrix h″ and performing beamforming in a specific direction as in Equation 2 and Equation 3.

### [Operation of Estimating Device 100]

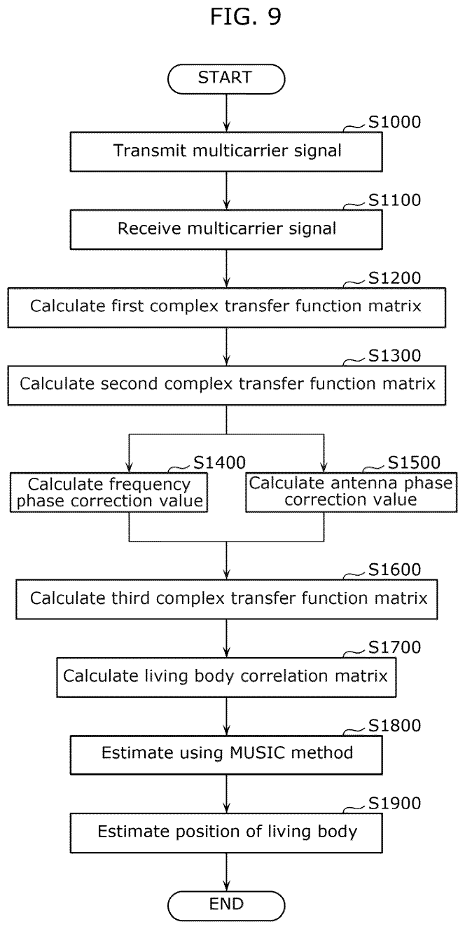

The operation in the estimation process by estimating device 100 configured in the above-described manner will be described. FIG. 9 is a flowchart illustrating the estimation process by estimating device 100 according to the present embodiment.

Estimating device 100 transmits a multicarrier signal including S subcarrier signals from M transmission antenna elements (S1000).

Next, estimating device 100 receives the multicarrier signal transmitted in step S1000 with one or more reception antenna elements (S1100).

Next, estimating device 100 calculates first complex transfer function matrix h from the received multicarrier signal (S1200).

Next, estimating device 100 performs correction of the asynchronous component on the calculated first complex transfer function matrix h, and calculates second complex transfer function matrix h′ (S1300).

Next, estimating device 100 calculates frequency phase correction value h_{cal1} based on the second complex transfer function matrix h′ (S1400).

Next, estimating device 100 calculates antenna phase correction value h_{cal2} based on the second complex transfer function matrix h′ (S1500). It should be noted that step S1400 may be performed before, after, or in parallel with step S1500.

Next, estimating device 100 calculates a third complex transfer function matrix h″ based on the calculated frequency phase correction value h_{cal1} and antenna phase correction value h_{cal2} (S1600).

Next, estimating device 100 extracts components related to the living body from the third complex transfer function matrix h″, to thereby calculate a living body correlation matrix having M×N rows and columns for each of the S subcarriers (S1700).

Next, estimating device 100 estimates third distance L and first angle θ using the MUSIC method, based on the living body correlation matrix (S1800).

Lastly, estimating device 100 estimates the position of living body 50 based on third distance L and first angle θ (S1900).

### Advantageous Effects, Etc.

According to the present embodiment, the coordinates of a living body can be estimated using an estimating device having a MISO or SIMO configuration.

As described above, the present disclosure can realize an estimating device and an estimating method that are capable of quickly and accurately estimating the distance or position of a living body by using radio signals.

Although a positioning sensor and an estimating method according to an aspect of the present disclosure has been described above based on exemplary embodiments, the present disclosure is not limited to these exemplary embodiments. Various modifications to the exemplary embodiments that can be conceived by a person of ordinary skill in the art or forms obtained by combining elements of different embodiments, for as long as they do not depart from the essence of the present disclosure, are included in the scope of the present disclosure.

For example, although estimation of the distance or position of living body 50 is described as an example in Embodiments 1 and 2, the present disclosure is not limited to living body 50. The present disclosure can be applied to various moving bodies (machines, etc.) whose activity imparts a Doppler effect on reflected waves in the case where a high-frequency signal is emitted.

In the above embodiments, each element may be configured using dedicated hardware, or may be implemented by executing a software program suitable for the element. Each element may be implemented by a program execution unit such as a CPU or processor reading and executing a software program recorded on a recording medium such as a hard disk or semiconductor memory.

The present disclosure can not only be realized as a positioning sensor including such characteristic elements, but can also be realized as an estimating method with steps corresponding to the characteristic elements included in the positioning sensor. The present disclosure can also be realized as a computer program that causes a computer to execute each of the characteristic steps included in such a method. It goes without saying that such a computer program can be distributed via a non-transitory computer-readable recording medium such as CD-ROM or via a communication network such as the Internet.

### INDUSTRIAL APPLICABILITY

The present disclosure can be used for positioning sensors and distance estimating methods that estimate the distance or position of a living body by using radio signals, and particularly, can be used for measuring instruments that measure the distance or position of a living body and a living body including a machine, home appliances that perform control according to the distance or position of a living body, distance measuring sensors mounted on surveillance devices that detect intrusion of a living body, direction estimating methods, and so on.

### REFERENCE SIGNS LIST

12 transmitter
13 transmission signal generator
21 reception antenna
22 receiver
50, 50-1, 50-2 living body
100 estimating device
111 transmission antenna
123 complex transfer function calculator
124 asynchronous component corrector
125 frequency phase corrector
126 antenna phase corrector
127 phase corrector
128 matrix transformer
129 living body correlation matrix calculator
130 estimator
131 positioner
1001 path from transmission antenna to living body
1002 path from reception antenna to living body
1101, 1102 phase change of complex transfer function matrix with respect to frequency
1202, 1201 phase of complex transfer function matrix after inverse Fourier transform
1203, 1203-1, 1203-2 ellipse
1301-A, 1301-B, 1301-C phase of each subcarrier signal transmitted from transmission antenna
1302-B, 1302-C phase change of signals with different frequencies transmitted from transmission antenna

1. An estimating device that estimates a distance to a living body and an angle, the estimating device comprising:
a transmission signal generator that generates a multicarrier signal obtained by modulating S subcarrier signals, where S is a natural number greater than or equal to 2;
a transmission antenna including M transmission antenna elements, where M is a natural number greater than or equal to 1;
a transmitter that causes the transmission antenna to transmit the multicarrier signal, by processing and outputting the multicarrier signal to the transmission antenna;
a reception antenna including N reception antenna elements, where N is a natural number greater than or equal to 1, where at least one of M or Nis greater than or equal to 2;
a receiver that measures, for a first period equivalent to a cycle derived from an activity of the living body, a plurality of reception signals which are received by each of the N reception antenna elements and include a reflected signal which is the multicarrier signal transmitted from each of the M transmission antenna elements that has been reflected or dispersed by the living body;
a first complex transfer function calculator that calculates, for each of S subcarriers to which the S subcarrier signals correspond, a plurality of first complex transfer functions indicating a propagation characteristic between a transmission antenna element and a reception antenna element in each of M×N combinations which are combinations of each of the M transmission antenna elements and each of the N reception antenna elements, using the plurality of reception signals measured by the receiver in the first period, and records the plurality of first complex transfer functions sequentially in time series which is an order in which the plurality of reception signals are measured;
a second complex transfer function calculator that calculates a second complex transfer function by dividing all elements based on the plurality of first complex transfer functions by a direct wave component extracted using one or more elements based on the plurality of first complex transfer functions, the direct wave component having arrived not via the living body from the plurality of reception signals;
a third complex transfer function calculator that calculates, from the second complex transfer function, a third complex transfer function by correcting, with respect to a reference phase calculated from a positional relationship between the M transmission antenna elements and the N reception antenna elements, an frequency phase error in the S subcarriers of the second complex transfer function, and an antenna phase error in the M transmission antenna elements;
a living body correlation matrix calculator that calculates a living body correlation matrix having M×N rows and columns for each of the S subcarriers, by extracting a component related to the living body from the third complex transfer function; and
an estimator that estimates, using the living body correlation matrix calculated for each of the S subcarriers, a third distance that is a sum of a first distance between the transmission antenna and the living body and a second distance between the reception antenna and the living body, and a first angle that is a direction of the living body as seen from the transmission antenna or the reception antenna.

2. The estimating device according to claim 1, wherein
the third complex transfer function calculator includes a frequency phase corrector that calculates a frequency phase correction value for correcting a frequency phase error in the S subcarriers for each of M×N propagation paths between the M transmission antenna elements and the N reception antenna elements, based on (i) ideal complex transfer functions in each of the M×N propagation paths obtained based on M×N inter-antenna distances between each of the M transmission antenna elements and each of the N reception antenna elements, and (ii) a reference complex transfer function matrix including M×N complex transfer functions measured in a second period, and corrects complex transfer functions for the first period.

3. The estimating device according to claim 1, wherein
the third complex transfer function calculator includes an antenna phase corrector that calculates an antenna phase correction value for correcting an antenna phase error in the M×N combinations for each of the S subcarriers, based on (i) ideal complex transfer functions in each of M×N propagation paths between the M transmission antenna elements and the N reception antenna elements obtained based on M inter-antenna distances between the M transmission antenna elements and the N reception antenna elements, and (ii) a reference complex transfer function matrix including M×N complex transfer functions measured in a second period, and corrects complex transfer functions for the first period.

4. The estimating device according to claim 1, further comprising:
a matrix transformer that transforms the third complex transfer function including M×N×S elements into a complex transfer function vector with dimensions of A×1 or 1×A, where A is an integer greater than or equal to 2 and less than or equal to M×N×S, wherein
the living body correlation matrix calculator calculates the living body correlation matrix having M×N rows and columns for each of the S subcarriers based on the complex transfer function vector with dimensions of 1×A.

5. The estimating device according to claim 1, wherein
the estimator estimates the third distance and the first angle using any one of a multiple signal classification (MUSIC) method, a Capon method, or a beamformer method.

6. An estimating method of estimating a distance to a living body and an angle, the estimating method comprising:
generating a multicarrier signal obtained by modulating S subcarrier signals, where S is a natural number greater than or equal to 2;
causing a transmission antenna to transmit the multicarrier signal, by processing and outputting the multicarrier signal to the transmission antenna, the transmission antenna including M transmission antenna elements, where M is a natural number greater than or equal to 1;
measuring, for a first period equivalent to a cycle derived from an activity of the living body, a plurality of reception signals which are received by each of N reception antenna elements included in a reception antenna and which include a reflected signal which is the multicarrier signal transmitted from each of the M transmission antenna elements that has been reflected or dispersed by the living body, where N is a natural number greater than or equal to 1, where at least one of M or N is greater than or equal to 2;
calculating, for each of S subcarriers to which the S subcarrier signals correspond, a plurality of first complex transfer functions indicating a propagation characteristic between a transmission antenna element and a reception antenna element in each of M×N combinations which are combinations of each of the M transmission antenna elements and each of the N reception antenna elements, using the plurality of reception signals measured in the first period, and recording the plurality of first complex transfer functions sequentially in time series which is an order in which the plurality of reception signals are measured;
calculating a second complex transfer function by dividing all elements based on the plurality of first complex transfer functions by a direct wave component extracted using one or more elements based on the plurality of first complex transfer functions, the direct wave component having arrived not via the living body from the plurality of reception signals;
calculating, from the second complex transfer function, a third complex transfer function by correcting, with respect to a reference phase calculated from a positional relationship between the M transmission antenna elements and the N reception antenna elements, a frequency phase error in the S subcarriers of the second complex transfer function, and an antenna phase error in the M transmission antenna elements;
calculating a living body correlation matrix having M×N rows and columns for each of the S subcarriers, by extracting a component related to the living body from the third complex transfer function; and
estimating, using the living body correlation matrix calculated for each of the S subcarriers, a third distance that is a sum of a first distance between the transmission antenna and the living body and a second distance between the reception antenna and the living body, and a first angle that is a direction of the living body as seen from the transmission antenna or the reception antenna.

7. A non-transitory computer-readable recording medium having recorded thereon a computer program for causing a computer to execute the estimating method according to claim 6.
